(19) United States
(12) Patent Application Publication
(10) Pub. No.: US 2014/0010859 A1
(43) Pub. Date: Jan. 9, 2014

(54) BARLEY PROTEIN MICROCAPSULES

(71) Applicant: Lingyun Chen, Edmonton (CA)

(72) Inventor: Lingyun Chen, Edmonton (CA)

(73) Assignee: THE GOVERNORS OF THE UNIVERSITY OF ALBERTA, Edmonton, AB (CA)

(21) Appl. No.: 13/992,497

(22) PCT Filed: Dec. 12, 2011
(86) PCT No.: PCT/CA2011/001355

§ 371 Date: Aug. 8, 2013

Related U.S. Application Data
(60) Provisional application No. 61/421,936, filed on Dec. 10, 2010.

(51) Int. Cl. A61K 9/50; A61K 31/015; A61K 45/00; A61K 38/22; A61K 38/18

(52) U.S. Cl. CPC A61K 9/5052; A61K 38/22; A61K 38/18; A61K 45/00; A61K 31/015
USPC 424/439; 514/9.7; 514/7.6; 514/763

(57) ABSTRACT

The invention is directed to microcapsules, pharmaceutical or nutraceutical compositions comprising same, and methods for preparing and using same for delivery of nanoparticle coated emulsions comprising biologically active ingredients. The microcapsule comprises barley protein, oil, and a biologically active ingredient. The microcapsule is degradable to generate a nanoparticle comprising an oil droplet coated with a barley protein.

### FIELD OF THE INVENTION

The invention relates to the preparation and use of barley protein microcapsules.

### BACKGROUND OF THE INVENTION

Nanoparticle coated emulsion droplets as drug carriers have attracted interest due to several advantages, including simple production methods at ambient temperature, the avoidance of organic solvents, and the high stabilization of poorly water soluble drugs in the hydrophobic domain of the internal oil core. Microfluidizers and high pressure homogenizers have been developed to “top-down” the particle size to between 30 nm to 100 nm. This is thought to be desired in order to exploit enhanced adherence or uptake by intestinal mucosa. Nanoparticle coated emulsion droplets offer much promise to stabilize and control drug release from emulsions compared to traditional submicron oil-in-water emulsions stabilized by surfactants and/or polymers. Such systems may thus be engineered to facilitate a range of release behaviors and have potential for oral delivery of poorly water-soluble drugs or nutraceuticals such as lipophilic vitamins, carotenoids, co-enzyme Q10, and the like.

However, nanoparticles, including nanoparticle coated emulsions, are usually prepared in an aqueous environment. Nanoparticles have thermodynamic driven tendencies to lower their interfacial surface area with the environment and to aggregate, leading to deterioration of their functionalities. Strategies for preventing aggregation have been adopted from conventional colloid science in which particles are coated with foreign capping agents and/or the surface charges are tailored to separate them via electrostatic repulsions. As an example, PEGylated nanoparticles have been developed to increase in vitro stability due to a steric stabilization mechanism. Additionally, a broad range of surfactants have been investigated in attempt to improve the stability of solid lipid nanoparticles during storage, the drug release profile, or the enzymatic degradation rate. Despite various surface modifications, the shelf life of nanoparticle suspensions is often limited. Moreover, once released into the human gut environment, the stability of the nanoparticles is largely impacted by pH, protease in the gut, and the presence of other compounds.

Microencapsulation has been widely used to protect fish oil from oxidation by forming an impermeable barrier to oxygen diffusion (Shu et al., 2006). This barrier also masks fish oil's unpleasant taste, and also creates a free flowing ‘dry’ powder to improve consumer acceptability and ease of handling (Barrow et al., 2009; Curtis et al., 2008). The physico-chemical properties of the microcapsule wall material are critical in governing the functionality of microcapsule systems (Gharsallaoui et al., 2007). Carbohydrates such as starches, maltodextrins and corn syrup solids are often used as microencapsulating agents due to their desirable drying properties and ability to form matrices (Gharsallaoui et al., 2007). However, carbohydrates usually have poor interfacial properties and must be chemically modified to improve their surface activity (Kanakdande et al., 2007; Krishnan et al., 2005; Soottitantawat et al., 2005). In recent years, an increasing interest in food protein-based microencapsulation can be attributed to their excellent emulsifying, gel- and film-formation properties (Chen et al., 2006). Additionally, protein coatings are degradable by digestive enzymes, thus can be used in developing food applications for controlled-core release (Chen et al., 2006). Whey proteins, caseinate and gelatins are the most common coating materials used to encapsulate fish oil by spray drying, spray cooling and coacervation methods. Spray drying is most commonly used in the food industry due to its continuous nature and adaptability to industrialization (Gharsallaoui et al., 2007; Gibbs et al., 1999; Gouin, 2004; Shu et al., 2006). The spray drying process normally involves an initial emulsification step, in which the protein wall material acts as a stabilizer for the core lipid. Next, the emulsion is converted into a free-flowing powder by spray-drying. Emulsions are typically solidified by adding a cross-linking reagent (e.g. transglutaminase), or coacervating with oppositely charged polysaccharides before spray-drying to reinforce the microcapsule structure. Whereas most research now has focused on animal proteins (Curtis et al., 2008; Kagami et al., 2003; Keogh et al., 2001; Subirade & Chen, 2008), little attention has been paid to plant proteins.

There is thus a need in the art for improved microencapsulation methods and delivery systems, utilizing plant proteins.

### SUMMARY OF THE INVENTION

The present invention relates to microcapsules comprising barley protein, pharmaceutical or nutraceutical compositions comprising same, and methods for preparing and using microcapsules for delivery of biologically active ingredients.

In one aspect, the invention comprises a microcapsule comprising a coating layer comprising barley protein, and an oil. The microcapsule may have a size between about 3 μm to about 5 μm in diameter, an encapsulation efficiency ranging between about 90% to about 100%, or a loading efficiency ranging between about 45% to about 50%.

In one embodiment, the microcapsule coating may consist essentially of hordein, consist essentially of glutelin, or may comprise hordein and glutelin. In one embodiment, the ratio of hordein to glutelin may be chosen in a pre-selected ratio.

In one embodiment, the oil may comprise a nut oil, or a vegetable oil, or a fish oil. The oil may further comprises a biologically active ingredient, which may be, for example, an antibiotic, antiviral agent, non-steroidal anti-inflammatory drug, analgesic, hormone, growth factor, vitamin precursor, or vitamin.

In another aspect, the invention may comprise a pharmaceutical or nutraceutical composition for treating, preventing or ameliorating a disease in a subject, providing a physiological benefit, or for providing protection from a chronic disease, comprising a microcapsule as described herein in combination with one or more pharmaceutically or nutraceutically acceptable carriers. The composition may be a food or beverage, such as a dairy product.

In another aspect, the invention may comprise a method of delivering a biologically active ingredient to a subject comprising administering to the subject in need thereof a microcapsule or a composition as described herein, wherein said microcapsule is degraded to smaller but intact nanoparticles in the stomach, and then more completely degraded in intestine. The delivery of the active ingredient may be indicated in a method of treating, preventing or ameliorating a disease in a subject.

In yet another aspect, the invention may comprise a method for preparing a protein encapsulated microcapsule, comprising the steps of:

a) blending an aqueous phase comprising barley protein and an oil to form a mixture;

b) emulsifying the mixture to form an emulsion; and

c) treating the emulsion to form microcapsules.

In one embodiment, the emulsion may be passed through a microfluidizer or a high pressure homogenizer to reduce the particle size in order to form microcapsules. In one embodiment, microcapsules may be dried, such as by the use of a spray dryer.

Additional aspects and advantages of the present invention will be apparent in view of the description, which follows. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The invention will now be described by way of an exemplary embodiment with reference to the accompanying simplified, diagrammatic, not-to-scale drawings:

FIGS. 1A and B are scanning electron microscopy images (SEM) showing the morphology of the barley protein microcapsules. FIG. 1C is a transmission electron microscopy (TEM) image showing the internal microstructure of the barley protein microcapsules.

FIGS. 2A, 2B and 2C are SEM images showing the morphology of spray dried BGH-2 microcapsules prepared at different inlet temperature: (a) 180° C., (b) 150° C. and (c) 120° C.

FIGS. 3A-3F are SEM images showing the Morphology of spray dried microcapsules with different wall components: (a) BH, (b) BGH-1, (c) BGH-2, (d) BGH-3, (e) BG and (f) inner structure of BGH-2.

FIG. 4 is a graph showing the release profile of β-carotene from barley protein microcapsules in simulated gastric (SGF) and intestinal (SIF) fluids.

FIG. 5 is a graph showing the degradation of barley protein microcapsules in SGF and SIF.

FIGS. 6A-D are TEM images of nanoparticle coated emulsions released after incubation in SGF for 30 minutes (FIG. 4A), 60 minutes (FIGS. 4B and C), and after incubation in SIF (FIG. 4D).

FIG. 7 is a photograph of a SDS-PAGE gel showing barley hordein (lane a), glutelin (lane b), hydrolyzed soluble protein after incubating barley protein microcapsules in SGF (lane c), and the protein layer coating on oil droplets (lane d).

FIG. 8 is a graph showing Peroxide value (PV) changes for encapsulated fish oil in dry status microcapsules with different wall components in accelerated storage test (40° C. for 8 weeks). Oil blank stands for non-encapsulated/crude fish oil.

FIGS. 9A and 9B are graphs showing Peroxide value (PV) changes for encapsulated fish oil in wet status microcapsules with different wall components during storage: (a) wet status microcapsules in pH 7.0 buffer; (b) wet status microcapsules in pH 2.0 buffer.

FIG. 10 is a graph showing Peroxide value (PV) changes for encapsulated fish oil in BGH-1 microcapsules in two food formulations (milk and yogurt).

### DETAILED DESCRIPTION OF PREFERRED EMBODIMENTS

When describing the present invention, all terms not defined herein have their common art-recognized meanings. To facilitate understanding of the invention, the following definitions are provided.

“Biologically active ingredient” means any biologically active compound such as a pharmaceutical including, for example, an antibiotic, antiviral agent, non-steroidal anti-inflammatory drug, analgesic, hormone, growth factor, vitamin precursor, vitamin, and the like, for use in the treatment, prevention, or amelioration of a disease. Biologically active ingredients useful in accordance with the invention may be used singly or in combination.

“Encapsulation efficiency” means the amount of oil encapsulated in the microcapsule divided by the amount of oil initially present in the loading solution, expressed as percentage.

“Loading efficiency” means the amount of oil encapsulated in the microcapsule divided by the amount of microcapsules, expressed as percentage.

“Microcapsule” means a microparticle ranging in largest dimension from about 0.1 μm and 100 μm, preferably from about 1 μm to 50 μm, more preferably from about 1 μm to 10 μm, and most preferably from about 3 μm to about 5 μm, which comprises an encapsulation coat and a core.

“Nanoparticle” means a particle having one dimension less than about 1000 nm, and preferably less than about 200 nm, and more preferably less than about 100 nm.

“Pharmaceutical effectiveness” or “pharmaceutical efficacy” means any desired pharmaceutical result.

“Pharmaceutically- or therapeutically effective amount” means a nontoxic but sufficient amount of the microcapsule composition to treat, prevent or ameliorate a condition of interest. For example, the term may refer to an amount sufficient to provide a desired response and corresponding therapeutic effect, or in the case of delivery of a therapeutic compound, an amount sufficient to effect treatment of the subject. The amount administered will vary with the condition being treated, the stage of advancement of the condition, the age and type of host, and the type and concentration of the formulation being applied. Appropriate amounts in any given instance will be readily apparent to those skilled in the art or capable of determination by routine experimentation.

“Pharmaceutically- or therapeutically-acceptable” is used herein to denote a substance which does not significantly interfere with the effectiveness or the biological activity of the active ingredient and which has an acceptable toxic profile for the host to which it is administered.

“Subject” means humans or other vertebrates.

“Zero-order release” means the delivery of a biologically active ingredient at a rate which is independent of time and the concentration of the active ingredient within a pharmaceutical dosage form. Zero order mechanism ensures that a steady amount of the active ingredient is released over time, minimizing potential peak/trough fluctuations and side effects, while maximizing the amount of time the active ingredient concentrations remain within the therapeutic window or efficacy.

The present invention relates to microcapsules comprising barley protein and oil, pharmaceutical or nutraceutical compositions comprising the same, and methods for preparing and using same for delivery of an oil. The oil may be the biologically active ingredient itself, or may comprise a biologically active ingredient, which is preferably oil-soluble. The microcapsules protect the oil and/or active ingredients upon encountering conditions which are incompatible. The oil and/or the biologically active ingredient may thereby be protected from mechanisms such as oxidation, inactivation through denaturation, damage, or degradation caused by heat, organic solvents, unfavorable pH, enzymes, and the like.

Barley (Hordeum vulgare L.) is grown primarily for animal feed and the brewing industry (Eagles et al., 1995), yet even after its use in brewing, the by-products become livestock feed. Barley grains and by-products are abundant and affordable protein sources which contain 8-13% and 20-30% (w/w) protein, respectably (Yalçin et al., 2008). Hordein and glutelin are the two major endosperm storage proteins of barley (35-55% and 35-40%, repectively), whereas albumin and globulin proteins are enriched in the bran and germ (Finnie & Svensson, 2009). The alcohol extracted hordein fractions can be further divided into five groups based on their electrophoretic mobility and amino acid compositions: B hordein (sulphur-rich), C hordein (sulphur-poor), γ-hordein (sulphur-rich), D hordein (high molecular weight), and A hordein (the smallest polypeptides) (Celus et al., 2006). B hordein (mol wt 35-46 kDa) and C hordein (mol wt 55-75 kDa) account for 70-90% and 10-30%, respectively, of the total hordein fraction (Shewry et al., 1983 & 1985). Glutelin is defined as an alkali-soluble protein after hordein extraction. But it is not possible to prepare a glutelin fraction totally free from hordein contamination (Celus et al., 2006). Both hordein and glutelin fractions are highly hydrophobic.

The present invention utilizes a microcapsule formed of barley protein which is capable of substantially protecting and stabilizing oil droplets which may comprise an oil-soluble active ingredient, upon exposure to acidic stomach pH and enzymes, and effectively delivering the microcapsule relatively intact to the small intestine. Embodiments of the barley protein microcapsules were characterized for their size, morphology, encapsulation efficiency, loading efficiency, stability, in vitro degradation and drug release as described herein.

In one embodiment, the microcapsules comprise barley protein-stabilized fish oil microcapsules in the order of 1-5 μm, which may be prepared by a pre-emulsifying process followed by a microfluidizer treatment. Stable solid particles were created in aqueous solution after microfluidizing, without the use of organic solvents or cross-linking reagents. In one embodiment, optimal conditions for microcapsule formation were 15% protein and a 1.0 oil/protein ratio. These microcapsules could be converted into free-flowing powders by a spray-drying process at an optimum inlet temperature of between about 120° C. to about 180° C., preferably between about 140° C. to about 160° C., and most preferably about 150° C. These microcapsules exhibited high oil encapsulation efficiency, loading efficiency, and low moisture content.

In one embodiment, a barley protein enriched in barley glutelin may provide for the maintenance of microcapsule coating integrity during spray-drying, to enable the formation of microcapsules with a dense and smooth surface. In another embodiment, a barley protein enriched in barley hordein conferred microcapsules with a comparably higher capacity to prevent oil oxidization. The proportion of glutelin to hordein may varied to provide microcapsules with desired characteristics.

In one embodiment, the invention comprises a method for preparing a microcapsule comprising the steps of:
a) pre-blending an aqueous phase comprising barley protein and an oil phase to form a mixture;
b) emulsifying the mixture to form an emulsion; and
c) treating the emulsion to produce microcapsules.
   In one embodiment, the emulsion may be treated with a microfluidizer or a high pressure homogenizer to reduce particle size and produce the microcapsules. In one embodiment, the microcapsules are dried, using for example a spray dryer, to create a flowable powder.

High-energy emulsification methods involve the introduction of mechanical shear through equipment such as high-shear stirrers, ultrasound generators, microfluidizers, and high-pressure homogenizers. High-pressure homogenizers are well known in the art, and have been widely used to prepare emulsions and submicron emulsions from bovine serum albumin, whey and soy protein.

To prepare conventional solid microcapsules, a protein stabilization step is normally conducted by using a protein cross-linking agent, changing the pH and temperature, or forming coacervates with an oppositely charged polysaccharide. In one embodiment of the present invention, exemplary microcapsules are prepared without a protein stabilization step. Once barley protein is extracted, it is blended into an aqueous phase and emulsified with an oil, preferably with high-pressure homogenization to form a first emulsion. In one embodiment, the first emulsion comprises oil droplets which also comprise an oil-soluble biologically active ingredient. The oil droplets may comprise one or more biologically active ingredient.

In an alternative embodiment, different populations of oil droplets may be admixed prior to use. Suitable oils include, but are not limited to, nut oils, and vegetable oils such as canola oil, corn oil, sunflower oil, safflower oil, sesame oil, soybean oil, peanut oil, palm oil, olive oil, coconut oil, rice bran oil, and the like, or a fish oil.

The size of the first emulsion is then reduced, preferably by using a microfluidizer or a high pressure homogenizer, to form the final product, namely the microcapsule of the present invention.

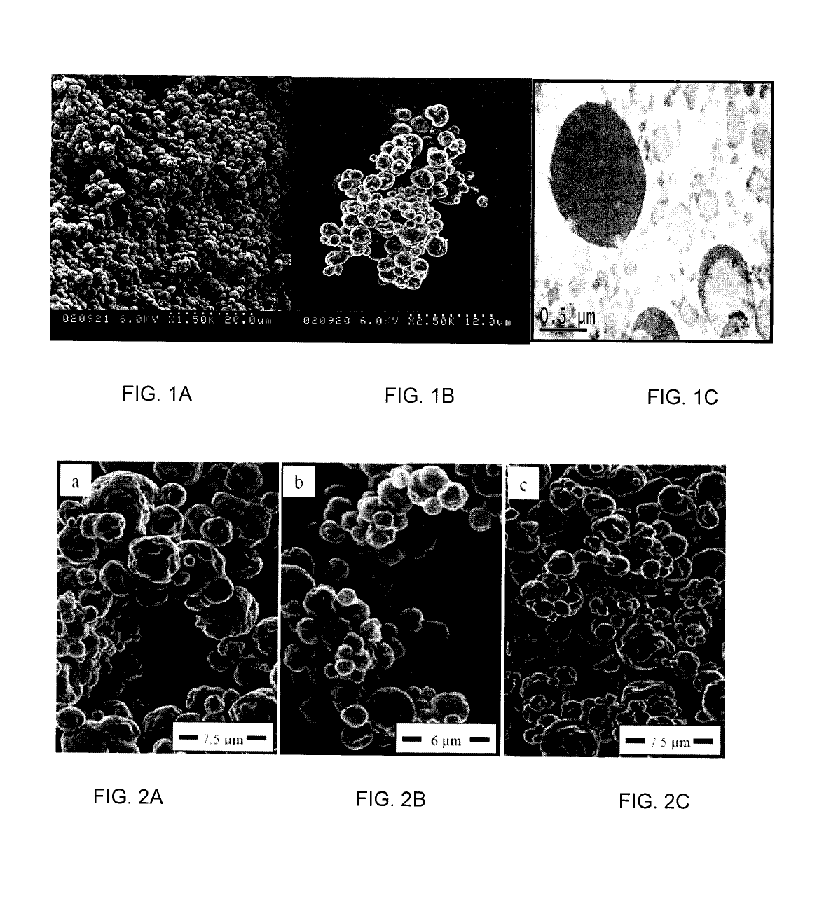

Exemplary barley protein microcapsules were confirmed to be spherical and smoothly surfaced, as determined by scanning electron microscopy (FIGS. 1A and 1B). In one embodiment, the size of the microcapsules ranges between about 3 μm to about 5 μm in diameter. In one embodiment, the size of the microcapsules is about 3.3 μm in diameter with a polydispersity index of 0.25. No aggregation was observed.

The internal morphology of the microcapsule was determined by transmission electron microscopy. In hydrophilic protein-stabilized emulsion systems, spherical oil droplets having smooth surfaces are homogenously distributed inside the matrices with a thin layer of protein aggregates around the oil droplets (data not shown). In contrast, barley protein may form a coating which fully covers the oil droplet or aggregates several oil droplets (FIG. 1C).

Barley protein has a unique structure with an abundance (approximately 40%) of non-polar amino acids on its side chains, and a conformation in which hydrophilic side chains are buried in the core and hydrophobic side chains are exposed outside of the core. Barley proteins are considered hydrophobic, which arises from barley protein molecular structures enriched with non-polar amino acids (˜35-38%) including proline, alanine, valine, isoleucine, and leucine (Wang et al., 2010). Hydrophobicity may enable barley proteins to rapidly adhere and completely cover oil droplets in the pre-emulsion process. They strongly aggregate to form thick unruptured coatings after microfluidizer treatment, with no need for cross-linking reagents or extra solidification processes. Without being bound by theory, the structure and conformation allow the barley protein to cover the oil droplets fully or aggregate droplets together, likely based on surface hydrophobic patches to form an un-ruptured coating.

In one embodiment, the microcapsules may be spray dried to turn wet-status microcapsules into dry status microcapsules in a flowable powder.

The payload capacity of the barley protein microcapsules results in an encapsulation efficiency ranging between about 90% to about 100%. In one embodiment, the encapsulation efficiency is about 95.5±2.6%. In one embodiment, the loading efficiency ranges between about 45% to about 50%. In one embodiment, the loading efficiency is about 47.8±1.3%.

The barley protein microcapsules were evaluated in vitro for their effectiveness in releasing a biologically active ingredient. Barley protein microcapsules were loaded with β-carotene as a model active ingredient. β-carotene is the major dietary precursor of vitamin A and is widely distributed in plants. However, only a small proportion of the total amount of β-carotenoids found in fruits and vegetables is bioavailable (Pan et al., 2007; Rich et al., 2003a, 2003b; Wang et al., 2010). A strategy to improve absorption of β-carotenoids in vivo is thus desirable.

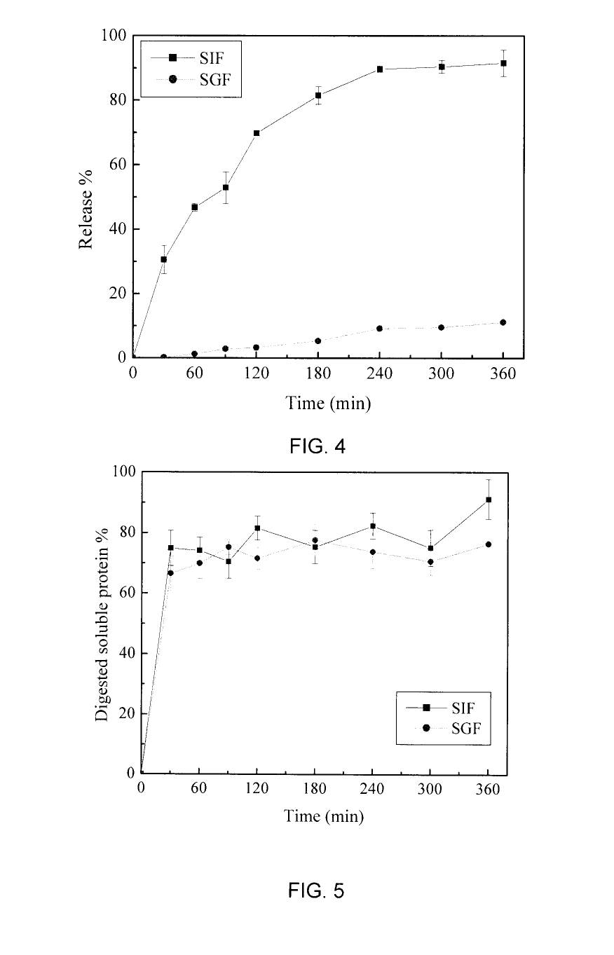

The release properties of the loaded barley protein microcapsules were determined in simulated gastric (SGF) and intestinal (SIF) fluids. FIG. 4 shows the profile of β-carotene release from barley protein microcapsules over time in simulated gastrointestinal tract fluids with and without digestive enzymes. Almost no β-carotene was released in gastric (pH 2.0) and intestinal (pH 7.4) fluid buffers.

Pepsin is an enzyme whose precursor form, pepsinogen, is released by the chief cells in the stomach and which degrades food proteins into peptides. When dispersed in SGF in the presence of pepsin, limited β-carotene release was observed. Five percent of β-carotene was detected in the release medium after two hours of the experiment, corresponding to the usual time for food to pass through the stomach to small intestine. This number increased to 10% only after six hours.

Pancreatin is a mixture of several digestive enzymes produced by the exocrine cells of the pancreas, and is composed of amylase, lipase and protease. In SIF, in the presence of pancreatin, β-carotene was steadily released from the barley protein microcapsules with near zero-order release kinetics (r²>0.991) in the first two hours. By that time, almost 70% of the β-carotene was released. The release curve then began to level off, with approximately 90% of the β-carotene being detected in the release medium after six hours. Without being bound by theory, near zero-order release in the small intestine may permit maximal utilization of an active ingredient in the body.

Release of active ingredients from protein matrices is commonly attributed to diffusion of the active ingredient or breakdown of the protein matrices, or both. In vitro protein matrix degradation was examined by suspending the barley microcapsules in SGF and SIF in the presence of digestive enzymes (FIG. 5). Barley protein microcapsules rapidly degrade in both SGF+pepsin and SIF+pancreatin. Almost 70% of the protein was converted to soluble protein hydrolysates after 30 minutes of incubation. The degradation curves then began to level off in the following hours. No obvious difference was observed between these two curves. The results indicate that degradation of the protein matrices played a major role in regulating β-carotene release from barley protein microcapsules since no release was observed in SGF and SIF without digestive enzymes. However, the much slower O-carotene release in SGF compared to SIF could not be explained by the degradation results, but may be explained by examining changes in morphology in SGF.

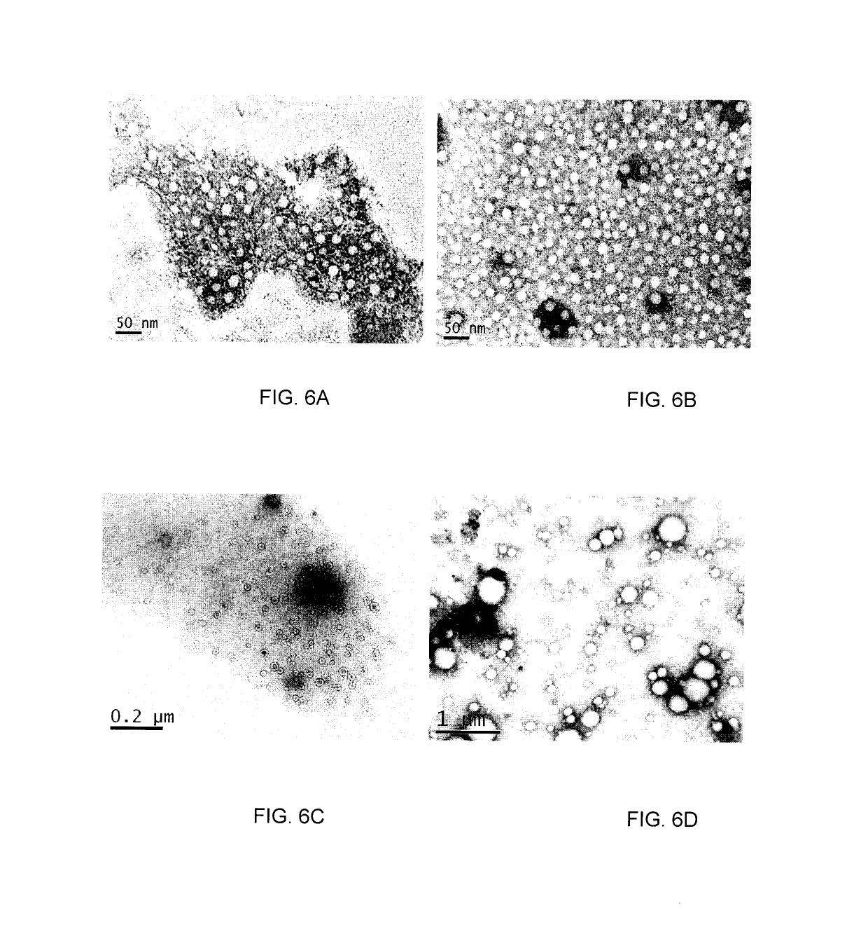

The morphology changes of the barley microcapsules incubated in SGF and SIF were observed using transmission electron microscopy. Nanoparticles having a size ranging from about 20 nm to 30 nm appeared as a result of microcapsule bulk matrices degradation when incubated in SGF for thirty minutes (FIG. 6A). Within one hour of incubation, bulk matrices disappeared, with near mono-dispersed nanoparticles remaining in the release medium (FIG. 6B). These nanoparticles have a core-shell structure featuring a solid protein coating (light part) on oil droplets (dark part).

The changes in the size of the microcapsules were also verified by Zetasizer™ analysis. A unimodal distribution with a peak in the 3.3 μm range was obtained for these microspheres when suspended in deionized water. After incubating 15 minutes in SGF with pepsin, a bimodal distribution was observed, with another peak appearing in the 50 nm range. The new peak corresponds to the nanoparticles which form after degradation of the microspheres. The smaller size observed in TEM compared to the Zetasizer™ may be attributed to shrinkage of the nanoparticles during air drying before TEM observation. Upon increasing the time to one hour, the peak corresponding to the 3.3 μm microcapsules disappeared and the peak in the 50 nm range increased simultaneously. These results confirm the degradation of the barley protein microcapsule matrices and release of the nanoparticles in SGF.

The stability of the nanoparticles was determined in order to confirm whether the nanoparticles may be transferred into the small intestine without aggregation. Stability was examined in simulated intestinal buffer (pH 7.4) without digestive enzymes. The released nanoparticles remained well-dispersed in buffer (pH 7.4) within 15-30 minutes, as observed by transmission electron microscopy (FIG. 6C). Some aggregation did occur after 30 minutes to 2 hours of incubation in intestinal buffer; however, most of the particles exhibited sizes ranging from about 50 nm to about 200 nm (FIG. 6D). These results suggest that the majority of the nanoparticles may be transferred intact into the small intestine.

The degradation of the released nanoparticles and the barley protein microcapsules was further examined in SIF in the presence of pancreatin, as observed using transmission electron microscopy. In contrast to the results observed in SGF, both particle matrices completely disappeared after one hour of incubation (data not shown), indicating that the protein was degraded by pancreatic enzymes. Without being bound by theory, the protein coating directly contacting the oil droplets may resist pepsin degradation to stabilize the oil droplets, which would account for the limited β-carotene release in SGF+pepsin. When transferred into the small intestine, the protein coating of the nanoparticles may be hydrolyzed gradually in the small intestine to release the active ingredient. Presumably the structure of the protein coating may be more important than the thickness of the adsorbed protein coating in providing resistance against hydrolysis. To enable structural characterizations, the protein coating was separated from the other digested proteins by precipitating the nanoparticles using ultracentrifugation.

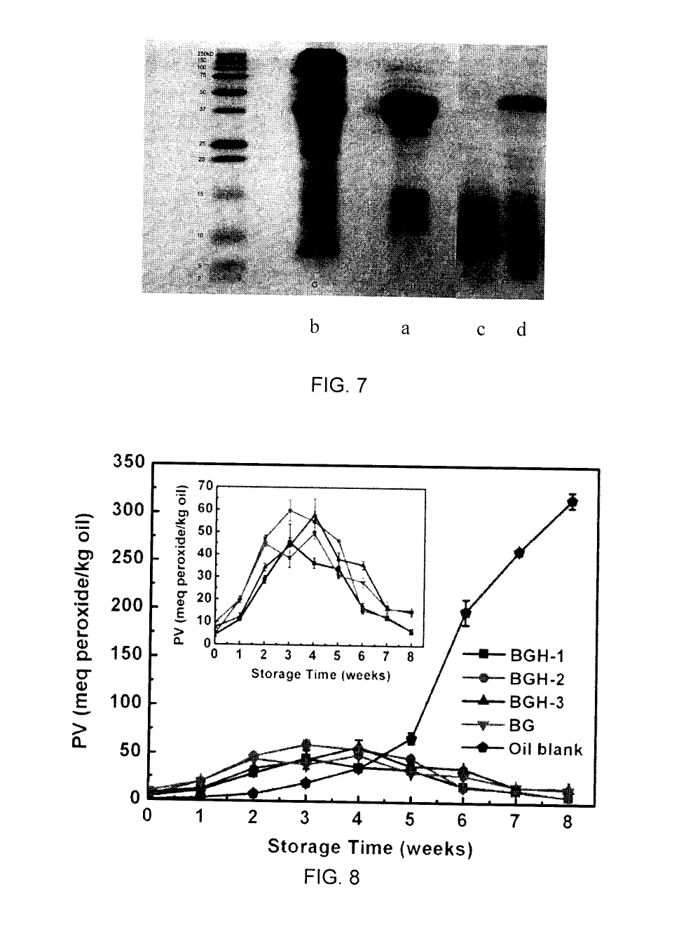

The proteins of the original barley protein fractions and the hydrolyzed soluble protein were separated on SDS-PAGE (FIG. 7). The barley protein was mainly composed of two fractions, glutelin and hordein. Glutelin is a simple, heat-labile protein found in the seeds and is soluble in dilute acids or bases. Hordein is a barley prolamin extracted with alcoholic media, comprises approximately 35-55% of the total barley grain protein, and is the main storage protein for barley. Barley hordeins are divided into four groups based on their electrophoretic mobilities and amino acid compositions: the B (30-50 kDa, sulfur-rich) and C (55-80 kDa, sulfur-poor) hordeins (70-80% and 10-20% of the hordein fraction, respectively) and the D (80-90 kDa) and A (15 kDa) hordeins (less than 5% of the total hordein fraction) (Kreis and Shewry, 1989; Davies et al., 1993; Celus et al., 2006). The A hordeins are likely alcohol-soluble albumins or globulins, or breakdown products of larger hordeins rather than true hordeins. C and some B hordeins appear as monomers, while most B and D hordeins are linked by inter-chain disulfide bridges.

Three subunits of hordein (lane a) were identified with bands at 55-80, 30-50 and <15 kDa corresponding to C, B and A hordeins, respectively. The barley glutelin showed four major bands at 85-90, 35-55, 20-25, <20 kDa (lane b). The 85-90 kDa band likely represents D-hordeins. The broad band at 35-55 kDa may be contamination of B hordeins in the glutelin fraction because it is not yet possible to prepare an undenatured glutelin fraction totally free of contaminating hordein. Following incubation in SGF for two hours, all the major bands disappeared, and broad bands appeared, indicating that the protein was hydrolyzed to ≦2 kDa (lane c). The SDS-page pattern of the protein coating the oil droplets showed two clear bands at 40-50 kDa (lane d), which may represent subunits of B-hordeins or peptides resulting from partially hydrolyzed C or D-hordeins which were resistant to pepsin digestion in SGF.

The amino acid composition of the protein coating was analyzed and compared with known amino acid compositions of B, C and D-hordeins and barley glutelin (Example 9) (Wang et al., 2010). As shown in Table 1, the protein coating has high glutamic acid (34.75%) and proline (29.15%), but low cysteine (0.37%).

TABLE 1
Amino acid composition of the isolated protein
layer coating on the oil droplets

| Residue | Content of Residue (%) |
|---|---|
| Asx | 3.57 |
| Ser | 4.35 |
| Glx | 34.75 |
| Gly | 4.44 |
| His | 0.78 |
| Arg | 1.83 |
| Thr | 2.65 |
| Ala | 2.37 |
| Pro | 29.15 |
| Cys | 0.37 |
| Tyr | 3.85 |
| Val | 2.09 |
| Met | 0.30 |
| Lys | 1.09 |
| Ile | 4.11 |
| Leu | 3.79 |
| Phe | 0.52 |

C-hordein peptide could be a major portion of the protein coating due to sharing a similar amino acid composition. C-hordeins consist almost entirely of an octapeptide repeat motif (consensus Pro-Gln-Gln-Pro-Phe-Pro-Gln-Gln) with a Mr of about 40,000. The secondary structure consists of an equilibrium between β-reverse turns and poly-L-proline II-like structure; however, as the protein concentration is increased and the protein becomes a hydrated solid, the secondary structure was found to consist of β-reverse turn and intermolecular β-sheet structures. C-hordeins are conformationally mobile and can undergo structural changes in passing from solution to a hydrated solid, allowing adsorption of C-hordeins on a hydrophobic surface to form a single molecule layer as observed using atomic force microscopy (McMaster et al., 1999).

Without being bound by theory, C-hordeins appear to be more competitive than other barley protein subunits to adsorb on the hydrophobic oil droplets during microcapsule formation owing to their unique molecular conformational mobility. Upon adhering to an oil surface, they appeared to aggregate to form un-ruptured films to fully cover the oil droplet. In SGF, the bulk microcapsule matrices were rapidly degraded by pepsin. The C-hordein protein coatings on the nanoparticles were however resistant to pepsin digestion. The resistance of C-hordein to pepsin degradation may relate to its repetitive structure with a high content of proline residues (˜30%), inhibiting the hydrolysis of some peptide bonds by proteolytic enzymes. When adhered to oil droplets, C-hordeins form a thin film with the hydrophobic side chains in contact with the oil phase, and the hydrophilic side chains facing outside. Since pepsin preferentially attacks peptide bonds involving hydrophobic aromatic amino acids, the protein coating presented a less vulnerable substrate to pepsin digestion.

When transferred in SIF, the released nanoparticles remained well-dispersed within 30 minutes of incubation. Although some aggregation occurred afterwards, most of the particles exhibited a size ranging from about 50 nm to 200 nm. It is expected that these nanoparticles could adhere to the intestinal mucosa owing to their submicron size. This will potentially prolong the formulation residence time by decreasing intestinal clearance mechanisms and by increasing the formulation surface area, allowing the active ingredients to better interact with the biological support. Pancreatin could breakdown the C-hordein coating of the nanoparticles completely during four hours of incubation, resulting in release of the active ingredients in SIF for a better absorption.

A number of factors may affect the ability of barley proteins to function as coating materials, such as protein structure and concentration, proportion of dispersed and dispersion phases, and processing conditions.

A high protein concentration in a particle mixture normally facilitates protein-protein interactions to form thick and viscoelastic layers at the oil droplet surface to encapsulate lipophilic compounds (Hogan et al., 2001). A high oil/protein ratio generally leads to a high capsule carrying capacity. In one embodiment, a maximum protein concentration of 15% was achieved for barley protein microencapsulation. Further increasing protein concentration led to the formation of aggregated substances rather than well dispersed microcapsules. Microcapsule quality is affected by wall material content and oil/protein ratio (Table 1 below). Hordeins (BH) may form into good coarse emulsions only at oil/protein ratio ≧1.0 after homogenization treatment. Hordein tends to aggregate to form soft and viscous dough when dispersed in water, likely due to a strong surface hydrophobicity (Wang et al., 2010). Protein aggregation could be associated with a reduction in the emulsifying capacity of the hordein at an oil/protein ratio of 0.5. Increasing the oil/protein ratio ≧1.0, more protein molecules would have an orientation of hydrophilic groups towards water phase and hydrophobic groups towards oil phase due to an increased dispersed phase volume, thus preventing protein aggregation and allowing formation of coarse emulsions. After passing the microfluidizer, solid BH microcapsules (wet status) were formed at an oil/protein ratio of 1.0 to 2.0.

Barley glutelin (BG) microcapsule formation was unaffected by increasing the oil/protein ratio from 0.5 to 2. Further increase of oil/protein ratio (≧2.0) induced an apparently higher viscosity, likely due to a highly dispersed phase volume (Hogan et al., 2001), leading to clumping particulate substances.

The BH and BG microcapsules formed were then spray-dried. Due to their sticky nature, BH microcapsule powders tended to adhere to the drying chamber wall surface, whereas free flowing BG microcapsules formed at the oil/protein ratio range of 0.5-1.0. Optimized conditions for BG and BH microcapsule preparation of (15% protein concentration and an oil/protein ratio of 1.0) were used to create microcapsules with gluten and hordein mixtures at ratios of 1:2 (BGH-1), 1:1 (BGH-2) and 2:1 (BGH-3) (Table 1).

The spray drying inlet temperature is another major factor impacting microencapsulation since it influences the microcapsule morphology. FIGS. 2A, 2B and 2C shows SEM micrographs of the BGH-2 microcapsules prepared at three different inlet temperatures (120° C., 150° C., and 180° C.). Irregular shaped microcapsules with less uniform size were obtained at the inlet temperature of 180° C. (FIG. 2A). This may be due to rapid particle shrinkage during the early stage of the drying process (Shu et al., 2006). Such particle features suggest that a 180° C. inlet temperature may be too high for barley protein microsphere preparation since high drying rates, associated with small particles, usually lead to rapid wall solidification (Rosenberg & Sheu, 1996; Sheu & Rosenberg, 1998). Decreasing the inlet temperature to 150° C., microcapsules were obtained with a spherical shape and a more uniform size (1-5 μm) (FIG. 2B). Further decreasing the temperature to 120° C. caused the powder particles to agglomerate (FIG. 2C). This can be attributed to the relatively high water content in the particle wall material resulted from inefficient drying. Water can act as an efficient plasticizer to decrease the glass transition temperature of the microsphere matrix. At the glass transition temperature, surface droplet viscosity and the powder particle stickiness increase, leading to inter-particle bridge formation that finally causes caking and the particle collapse (Beristain et al., 2002; Drusch et al., 2006&2007; Le Meste et al. 2002, Partanen et al., 2005). Therefore, in one embodiment, the microcapsules are spray dried with an inlet temperature between about 120° C. and 180° C., and preferably between about 140° C. and 160° C.

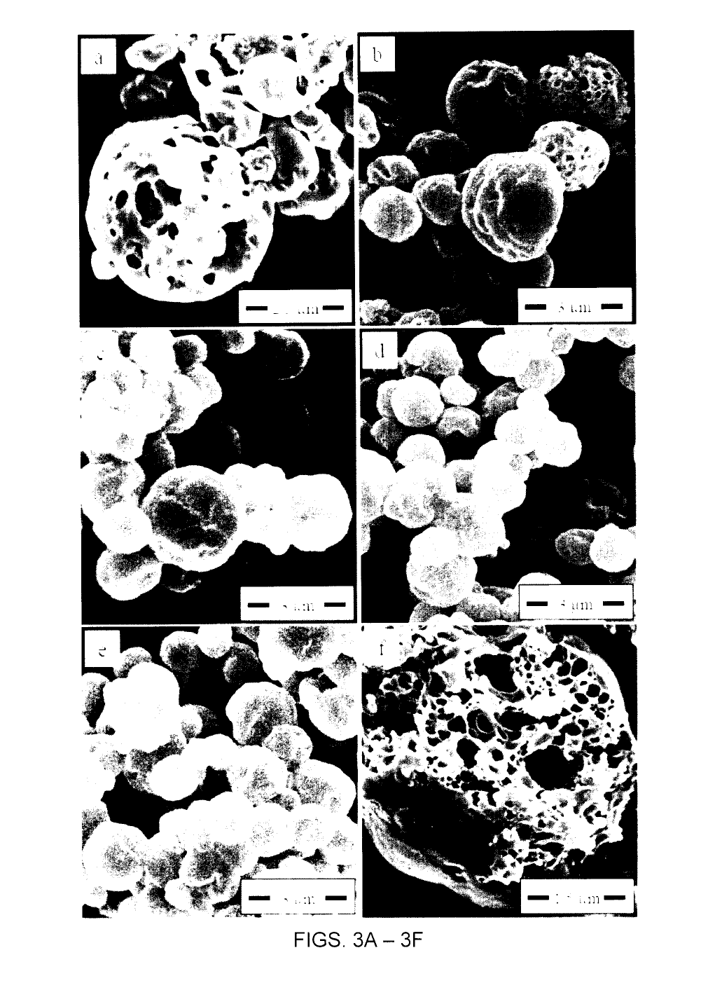

The spray-dried microcapsules were generally spherical in shape with diameters ranging from 1 to 5 μm as assessed by SEM (FIGS. 3A-3F). Similar results were obtained using Zetasizer (3.31±0.40 μm) for wet status microcapsules. This size range is typical for microcapsules intended for food applications. Although there were no significant differences in the diameters of microcapsules made from different protein fractions, their surface topographies differed. The presence of a surface porous microstructure was inversely related to the proportion of included glutelin in the wall material. BH and BGH-1 microcapsules exhibited a porous outer shell (FIGS. 3A and 3B), whereas BGH-2, BGH-3 and BG microcapsules demonstrated dense, crack-free and smooth surfaces (FIG. 2C-2E). During spray-drying, fast drying rates can lead to rapid hordein wall ballooning at an early stage of heating. This process can also be accompanied by hordein denaturation and the loss of viscoelasticity (Cauvain, 2003). This explains why further expansion resulted in the breaking of coating networks, leading to a more porous structure. BG did not exhibit viscoelastic characteristics, and therefore maintained a dense coating wall during the whole spray-drying process. BGH-2 and BGH-3 microcapsules exhibited similar surface morphologies as that of BG microcapsules, suggesting that the coating wall surface was mainly composed of glutelin, forming a dense external structure preventing hordein from ballooning. Therefore, in one embodiment, the addition of glutelin is important to maintaining microcapsule coating integrity during spray-drying.

FIG. 3F shows the inner structure of the BGH-2 microcapsules. Small pores were well distributed inside the BGH-2 matrix, likely representing smaller oil droplets that were originally present in the microcapsules. Such inner structure indicated that oil droplets were well distributed/separated within the protein micron-matrix. Other barley protein microcapsules showed similar porous inner structures (data not shown). The dense, crack-free surface features together with the interior multiple emulsion “honeycomb-like” structure, may confer barley protein microspheres the ability to better withstand mechanical stresses and protect the incorporated ingredients against harsh environments (e.g. oxidation, light, low or high pH).

Barley protein based wall materials were effective encapsulating agents as demonstrated by their high EE and LE values (Table 2).

TABLE 2
Encapsulation efficiency (EE), loading efficiency
(LE) and moisture content of the microcapsules

| Samples | EE | LE | Moisture |
|---|---|---|---|
| BH | 92.9 ± 1.7 | 46.5 ± 0.8 | NA |
| BG | 97.0 ± 2.9 | 48.5 ± 1.5 | 0.90 ± 0.017 |
| BGH-1 | 100.2 ± 2.1 | 50.1 ± 1.1 | 0.75 ± 0.032 |
| BGH-2 | 95.5 ± 2.6 | 47.8 ± 1.3 | 0.86 ± 0.064 |
| BGH-3 | 97.1 ± 2.2 | 48.6 ± 1.1 | 0.77 ± 0.070 |

Note:
NA means not available
Only a small amount of dried BH microcapsules were obtained for EE, LE and morphology analysis

Barley protein possesses excellent emulsifying properties (Wang et al., 2010) and a capacity to form solid microcapsule-coating-granule structures after microfluidizer or homogenizer treatment. In spite of the porous structure, BH microcapsules demonstrated slightly lower EE and LE values compared to other barley protein microcapsules (p<0.05), indicating that hordein may have the capacity to bind oil droplets and keep them inside the microcapsule matrix. Surface oil is an important indicator for microencapsulation evaluation; however, the normal methods used to determine microcapsule surface oil for other proteins (whey protein, caseinate, etc.) could not be used for barley protein microcapsules. Organic reagents (e.g. isohexane) that normally used to extract surface oil, extract both surface and encapsulated oil, likely due to barley protein's greater hydrophobicity.

The moisture content is also critical for formed microcapsules. High moisture will induce high viscosity and stickiness of powder particles, resulting in the formation of inter-particle bridges that lead to caking and particle collapse and the release/oxidation of the core material (Beristain et al., 2002; Drusch et al., 2006&2007; Le Meste et al. 2002, Partanen et al., 2005). In one embodiment, the moisture content of barley protein microcapsules, prior to any drying step, was maintained at relatively low levels, below about 2%, and preferably ranging from 0.75 to 0.90% (w/w). In contrast, published data for whey protein microcapsules moisture range from 2.24%-2.89% (Bae & Lee, 2008). The low moisture of barley protein microcapsules may be due to their hydrophobic nature, which would exclude water from the matrix. A slight decrease of moisture was observed with increasing of hordein content in the wall material (p<0.05). As an alcohol soluble protein, hordein has been reported to have higher percentage of non-polar amino acid groups compared to glutelin (Wang et al., 2010). Increasing hordein content may be an efficient way to decrease moisture in wall systems and thus reduce the chances of particle agglomeration and potential core oxidation.

The oxidative stability of encapsulated fish oil was analyzed under storage conditions of 40° C., because lower and ambient temperatures often require a long period of time. The tests were performed at a dry condition (using spray-dried microcapsules) as well as in pH 2.0 and 7.0 solutions (using freshly prepared microcapsules in wet status). The oxidation of unsaturated oil creates a variety of compounds including free radicals and hydroperoxides (Firestone, 1993). Peroxide value (PV) is a measure of the amount of hydroperoxide, representing the initial stage of fat and oil deterioration, and is a standard index to monitor food safety and quality. FIG. 8 shows the PV changes of the encapsulated fish oil in spray dried microcapsules (dry-status) at 40° C. during 8 weeks. Unencapsulated bulk fish oil was also tested as a control under the same conditions. Desirable stability was observed for oil blank (crude fish oil without any processing treatment containing no antioxidant) within 2 weeks of storage (<10 meq peroxide/kg oil), but the PV increased markedly after 5 weeks reaching a maximum level at almost 350 meq peroxide/kg oil in the 8th week. On the contrary, the PV values of fish oil encapsulated in barley protein microcapsules gradually increased and reached maximum levels of 45-76 meq peroxide/kg oil in the 3-4 weeks, and then declined to 6.6-15 meq peroxide/kg oil in the 8th week. The higher initial PV can be attributed to the oxidation of microcapsule surface/near surface oil during preparation when it was exposed to oxygen, light and heat. The auto-oxidation of encapsulated and non-encapsulated core likely occurs during the spray drying process catalyzing further oxidation in the subsequent storage test (Drusch & Berg, 2008; Drusch & Schwarz, 2006). The peroxides in oxidized oil are usually unstable and are themselves oxidized to other compounds. At the beginning of oxidation, peroxides increase but are eventually oxidized to aldehydes and ketones, explaining why the peroxide levels fall in the later stages (Drusch et al., 2006 & 2007; Firestone, 1993; Naohiro & Shun, 2006). After oxidation of surface/near surface oil, no further increase of the PV was detected in our result, suggesting the inside oil was well protected in the microcapsule matrix.

Among barley protein microcapsules, BGH-1 microcapsule matrices, with higher hordein content, had better protective ability. It has been reported that C hordeins possess superior antioxidative and reducing activity (Kawase et al., 1998; Wasaporn et al., 2009). As one major hordein fraction, C hordein consists almost entirely of repeats based on the octapeptide motif Pro-Gln-Gln-Pro-Phe-Pro-Gln-Gln and has demonstrated conformational transitions between poly-L-proline II-like and βI/III turn structures. The repetitive domain seems to form a helical secondary structure rich in β-turns and the entire molecule is rod-like with dimensions of about 30 nm×2 nm. Without restriction to a theory, such a unique structure may form a “cage” to better hold lipid molecules inside the protein matrix and protect it against oxidation. Additionally, the abundant hydrophobic amino acids (Leu, Val, Phe and Tyr) in the hordein fraction may also bind encapsulated oil contributing to its better oxidative stability (Wang et al., 2010).

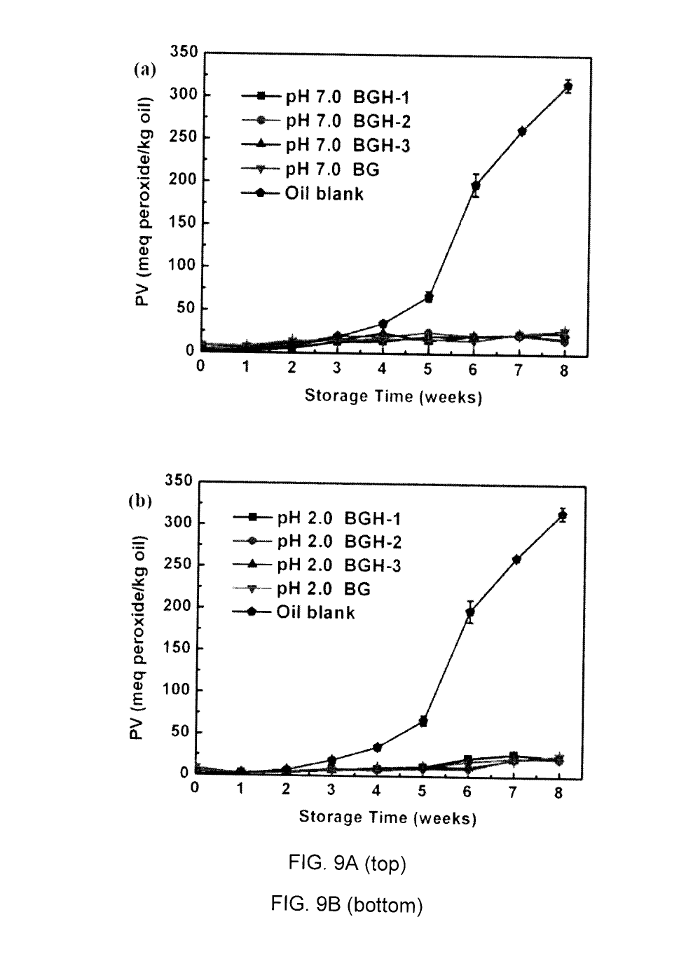

The PV level of fish oil encapsulated in freshly prepared microcapsules (wet status) was measured to evaluate the potential of using barley protein microcapsules in aqueous solutions. Buffers with a pH of 7.0 and 2.0 were chosen as representatives for neutral and acidic environments, respectively. No oil leakage was observed for any of the barley protein microcapsule suspensions after 8 weeks storage, indicating the integrity of microcapsules was well maintained. FIG. 9 shows the PV changes of fish oil encapsulated in wet status microcapsules at 40° C. for 8 weeks, at pH 7.0 and 2.0, respectively. All microencapsulated fish oil had low oxidative levels (PV<30 meq peroxide/kg oil) after 8 weeks of storage. No significant difference was observed for different matrixes in either pH 7.0 or 2.0 media (FIGS. 9A and 9B). This suggests barley protein microcapsules (wet-status) may be suitable for liquid/semi-liquid food applications. The much lower PV level for wet status compared to that of dry status confirms dry status lipid oxidation may be initiated by the spray-drying process. This drying process may lead to leakage of encapsulated oil to the exterior of the microcapsules, ultimately resulting in the acceleration of oxidative changes and a higher PV.

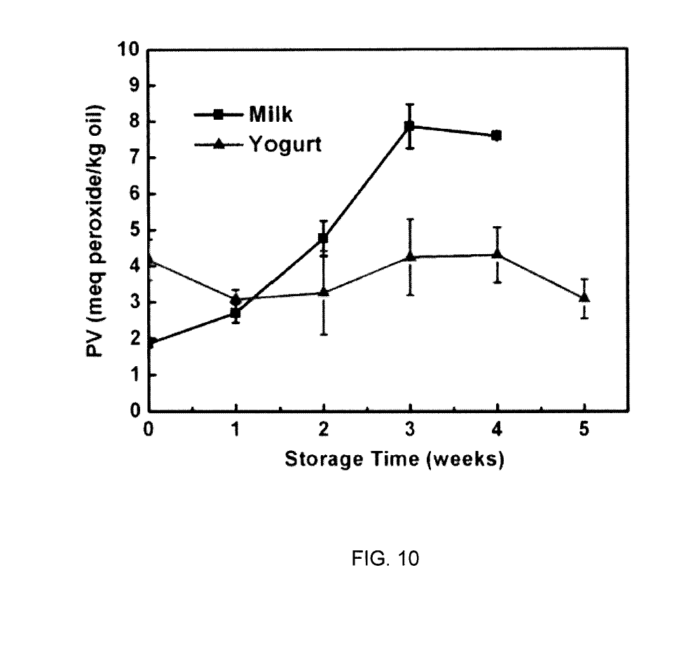

The microcapsules of the present invention may be formulated into food products, such as dairy products, for example. Wet status microcapsules were added in fat free milk and yogurt. The PV of encapsulated fish oil was measured weekly for milk and yogurt at 4 and 5 weeks, respectively, corresponding to their average shelf life. Both were pasteurized (80° C., 30 min) before storage (Ng et al., 2011) but after enrichment with microcapsules. As shown in FIG. 10, the PV of encapsulated fish oil remained low (PV<10 meq peroxide/kg oil) in both milk and yogurt during their storage. The fish oil microcapsules were especially stable in yogurt with PV levels below 5 meq peroxide/kg oil even after 5 weeks, well below the recommended PV levels (less than 30 meq peroxide/kg oil) in an edible food product (Naohiro & Shun, 2006).

Thus, microcapsules of the present invention can be used to deliver a wide variety of oils and/or biologically active ingredients to a subject, and hence may be used to treat, prevent, or ameliorate diseases, or to provide a physiological benefit, or may provide protection against a chronic disease. In one embodiment, the barley protein microcapsule of the invention can be used for site-specific targeted delivery, particularly to the small intestine. As used herein, “treatment” refers to the prevention of infection or reinfection, the reduction or elimination of symptoms, or the reduction or substantial elimination of a pathogen or a disease, or disorder. Treatment may be effected prophylactically or therapeutically.

The microcapsule may be present as a population of microcapsules in the form of a pharmaceutical or nutraceutical composition. In one embodiment, the invention is directed to a composition for treating, preventing, or ameliorating a disease comprising barley protein microcapsules in combination with one or more pharmaceutically acceptable fluids or carriers. Those skilled in the art are familiar with any pharmaceutically acceptable carrier that would be useful in this regard, and therefore the procedure for making pharmaceutical compositions in accordance with the invention will not be discussed in detail. Suitably, the pharmaceutical or nutraceutical compositions may be in the form of tablets, capsules, liquids, lozenges, lotions, aerosol, and solutions suitable for various routes of administration including, but not limited to, topically, orally, via injection or infusion, intraperitoneally, nasally, or rectally, in solid, semi-solid or liquid dosage forms as appropriate and in unit dosage forms suitable for easy administration of fixed dosages.

As used herein, physiologically acceptable fluid refers to any fluid or additive suitable for combination with a composition containing barley protein microcapsules. Typically these fluids are used as a diluent or carrier. Exemplary physiologically acceptable fluids include but are not limited to preservative solutions, saline solution, an isotonic (about 0.9%) saline solution, or about a 5% albumin solution or suspension. It is intended that the present invention is not to be limited by the type of physiologically acceptable fluid used. The composition may also include pharmaceutically acceptable carriers. Pharmaceutically accepted carriers include but are not limited to saline, sterile water, phosphate buffered saline, and the like. Other buffering agents, dispersing agents, and inert non-toxic substances suitable for delivery to a subject may be included in the compositions of the present invention. Adjuvants may be added to enhance the pharmaceutical effectiveness of the composition. The compositions may be solutions, suspensions or any appropriate formulation suitable for administration, and are typically sterile and free of undesirable particulate matter. The compositions may be sterilized by conventional sterilization techniques.

In one embodiment, the invention comprises a method of delivering a biologically active ingredient to a subject comprising administering to the subject in need thereof, the above microcapsule or the above pharmaceutical composition.

In one embodiment, the invention comprises a method of treating, preventing or ameliorating a disease in a subject, or providing a physiological benefit, or protection against a chronic disease, comprising administering to the subject in need thereof, a therapeutically effective amount of the above microcapsule or the above pharmaceutical composition.

Exemplary embodiments of the present invention are described in the following Examples, which are set forth to aid in the understanding of the invention, and should not be construed to limit in any way the scope of the invention as defined in the claims which follow thereafter.

### Example 1

### Materials

Regular barley grains (Falcon) were kindly provided by Dr. James Helm, Alberta Agricultural and Rural Development, Lacombe, Alberta. Protein content was 13.2% (w/w) as determined by combustion with a nitrogen analyzer (Leco Corporation, St. Joseph, Mich., USA) calibrated with analytical reagent grade EDTA. A factor of 6.25 was used to convert the nitrogen to protein. Canola oil used for the emulsification was purchased from a local supermarket (Edmonton, AB, Canada). Unstained standard protein molecule marker for SDS-PAGE was purchased from Bio-RAD (Richmond, Calif., USA). Beta-carotene, pepsin (from porcine gastric mucosa, 424 units/mg) and pancreatin (from porcine pancreas) were purchased from Sigma-Aldrich, Canada (Oakville, ON, Canada). Fish oil (Omega 30 TG Food Grade (Non-GMO) MEG-3™ Fish Oil) was kindly donated by Ocean Nutrition Canada Limited (ONC) (Canada) with (EPA+DHA) content ˜31%. Fat free yogurt (Yoplait Vanilla, Yoplait USA, Inc) and fat free milk (Lucerne skim, Safeway Inc) used for food formulation were purchased from a local grocery store. All other chemical reagents were purchased from Fisher Scientific (Ontario, Canada) and were used as received unless otherwise described. All other chemicals were of reagent grade.

### Example 2

### Barley Protein Extraction

Barley protein was extracted according to Wang et al. (2010). Briefly, after pearling and milling, barley endosperm flour was dispersed in an alkaline solution (pH 11) adjusted using 0.1M NaOH solution at a solvent-to-flour ratio of 10:1 (v/w) with stirring for 0.5 h at room temperature (23° C.). After extraction, the insoluble solids were separated by a centrifuge (Beckman Coulter Avanti™ J-E Centrifuge, Calif., USA) at 8,500×g for 15 min at 23° C. The supernatants were adjusted to approximately pH 5 with 0.5 M HCl to precipitate the proteins. Protein isolates were then obtained by centrifugation at 8,500×g for 15 min at 23° C. All isolated protein fractions were lyophilized and the dry powders were stored in plastic bags at 4° C. before further analysis. Protein content of the isolated barley protein fractions was determined using the Leco™ nitrogen analyzer (Leco™, St. Joseph, Mich., USA).

### Example 3

### Microcapsule Preparation

In a first example, premixed emulsion was prepared by mixing 15% (w/w) barley protein suspension as an aqueous phase with canola oil containing 0.05% (w/v) β-carotene (model bioactive compounds) at the ratio of 1:1 (w/w) using a PowerGen™ homogenizer (Fisher Scientific International, Tustin, Calif., USA). Finer microcapsules were then formed by passing the premixed emulsion through a microfluidizer (model M110-S; Microfluidics Corp, Newton, Mass., USA) operated at 350 bar homogenization pressure. To prevent an increase in the temperature of the final product, cold water was circulated at the outlet of the homogenizing valve. The prepared microcapsules in suspension were stored at 4° C. with 0.025% sodium azide until use.

In a second example, the barley protein powders were hydrated at pH 11.0 (adjusted with 3N NaOH) to form a 15% (w/v) solution. The pH was then adjusted to 7.0 followed by an immediate mixing with fish oil to form a coarse emulsion using a homogenizer (30,000 rpm/min) (PowerGen, Fisher Scientific International, Inc., CA, USA). Microcapsules were then formed by passing the premixed emulsion through a microfluidizer system (M-110S, Microfluidics Co., USA) operated at 350 bar. To prevent an increase in temperature of the final product, the pipe components of the microfluidizer was immersed in ice. The prepared microcapsules (wet status) were stored at 4° C. with 0.025% (w/v) sodium azide until further analysis.

Parts of the wet status microcapsules were spray dried using a lab scale spray dryer (Büchi 190 Mini Spray Dryer, Büchi Labortechnik, Flawil, Switzerland). Three different air inlet temperatures (180° C., 150° C. and 120° C.) were applied to study the impact of hot air on microcapsule morphology. The outlet temperature was controlled between 55-65° C. (Shu et al., 2006). The dried microcapsules (dry status) were stored in plastic bottles at 4° C. before analysis. The prepared samples were coded as shown in Table 1.

TABLE 1
Preparation of the barley protein microcapsules and their components

| | | Microcapsule | | | Formation of | Formation of |
|---|---|---|---|---|---|---|
| | | components (wt %) | | | wet | dry |
| Samples | | Fish Oil | Hordein | Glutelin | microcapsules | microcapsules |
| BH | Hordein | 33.3 | 66.7 | 0 | No | No |
| | | 50.0 | 50.0 | 0 | Yes | No |
| | | 66.7 | 33.3 | 0 | Yes | No |
| BG | Glutelin | 33.3 | 0 | 66.7 | Yes | Yes |
| | | 50.0 | 0 | 50.0 | Yes | Yes |
| | | 66.7 | 0 | 33.3 | Yes | No |
| BGH-1 | G:H = 1:2 | 50.0 | 33.3 | 16.7 | Yes | Yes |
| BGH-2 | G:H = 1:1 | 50.0 | 25.0 | 25.0 | Yes | Yes |
| BGH-3 | G:H = 2:1 | 50.0 | 16.7 | 33.3 | Yes | Yes |

### Example 4

### Microcapsule Characterizations

The particle size of the first example was measured at room temperature by dynamic light scattering using a Zetasizer NanoS™ (model ZEN1600, Malvern Instruments Ltd., UK). The microcapsule suspensions were diluted in deionized water to a suitable concentration before analysis. The protein refractive index (RI) was set at 1.45 and dispersion medium RI was 1.33. All data were averaged from at least three batches. The morphology observation of the microcapsules was carried out with a Hitachi™ X-650 scanning electron microscopy (SEM, Hitachi, Tokyo, Japan). The samples were freeze-dried before SEM observation. The cross-sections and surfaces of the gels were sputtered with gold, observed and photographed. The interior morphology of the microcapsules was also observed using transmission electron microscopy (TEM, Hitachi, Tokyo, Japan). The samples were immersed in propylene oxide, propylene oxide/Epon™ solution (1:1), and finally pure Epon. After infiltration overnight at room temperature, they were embedded in Epon, with polymerization at 60° C., thinly sectioned, stained with uranyl and lead acetate, and viewed at 100 kV.

The size of microcapsules in the second example in wet status was measured at room temperature by dynamic light scattering using a Zetasizer NanoS instrument (model ZEN1600, Malvern Instruments Ltd, UK). The protein refractive index (RI) was set at 1.45 and the dispersion medium RI was 1.33. The microcapsule suspensions were diluted in deionized water to a suitable concentration before analysis and data were averaged from at least three batches. The morphology of the spray-dried microcapsules was observed with a scanning electron microscope (SEM, S-2500, Hitachi, Tokyo, Japan) operating at 15 kV. The surfaces of the microcapsules were sputtered with gold, observed and photographed. The powders were also fractured carefully after frozen in liquid nitrogen, and the interior morphology was observed and photographed using the SEM.

### Example 5

### Beta-Carotene Encapsulation

To determine the payload capacity, spray dried microcapsules (200 mg) from the first example were precisely weighed and added into 5 ml pure ethanol followed by vortex mixing. 5 ml hexane was then added into this dispersion and vortex mixed. Finally 5 ml deionized water was added in this mixture by gentle shaking. The supernatant was then obtained by centrifuge at 8000×g for 15 min at room temperature. After evaporating the volatile solvent by blowing nitrogen gas, the remaining oil was precisely weighed. The encapsulation efficiency (EE) and loading efficiency (LE) were calculated by the following equations:

EE(%)=Amount of oil in microcapsule/Oil initially added×100 (1)

LE(%)=Amount of oil in microcapsule/Amount of microspheres×100 (2)

### Example 6

### In Vitro Release of Beta-Carotene

Beta-carotene release was determined by incubating wet microspheres (−240 mg in dry weight) in 24 ml of a release medium with continuous agitation by magnetic bar (100 rpm) at 37° C. The following four release media were used: HCl solution (pH 2.0); phosphate-buffered saline or PBS (pH 7.4); simulated gastric fluid (SGF) USP XXII (pH 2.0) with 0.1% pepsin (w/v); and simulated intestinal fluid (SIF) USP XXII (pH 7.4) with 1.0% pancreatin (w/v). One tube was withdrawn at every half hour or one hour interval. Hexane (5 ml) was used to extract the released β-carotene by vortex mixing. The β-carotene content in the hexane (sealed to avoid evaporation) was determined by measuring the absorbance at 450 nm with a UV-visible spectrophotometer (model V-530, Jasco, Calif., USA) (Pan et al., 2007).

### Example 7

### In Vitro Protein Matrix Degradation

In vitro protein matrix degradation was examined by suspending wet microcapsules in simulated gastric fluid (SGF) or intestinal fluid (SIF) under the same conditions as described in Example 6. After removing the released oil phase containing β-carotene by hexane, the solutions were heated to 95° C. for 3 min to inactivate the enzymes. The digested mixtures were then centrifuged at 18,000×g for 20 min at room temperature. The supernatants were filtered through a Whatman No. 1 filter paper to obtain clear filtrates. The protein concentration in the filtrates was determined by a Bradford dye assay with bovine serum albumin as the standard. The percent degradation was expressed as a percentage of the soluble protein content of the starting microcapsule sample. Blank SGF and SIF solutions were run as controls.

The morphology changes of the microcapsules incubated in SGF and SIF were also observed. The samples were prepared by coating a copper grid with a thin layer of digestive suspension. After negative staining with 1% (w/v) phosphotungstic acid, excess liquid was blotted from the grid. Samples were then air dried and examined using a TEM at an accelerating voltage of 100 kV. The particle size change during incubation in SGF was also monitored using the Zetasizer NanoS™ (model ZEN1600, Malvern Instruments Ltd, UK). The digestive suspensions were diluted in buffer (pH 2.0) to a suitable concentration before analysis. All data were averaged from at least three batches.

### Example 8

### Isolation of Protein Coating from Oil Droplets and SDS-PAGE

The digestive mixtures after incubation in SGF were isolated by centrifugation at 20,000×g for 15 min at room temperature. The precipitates were collected and washed thoroughly with deionized water. The protein layer coating on oil droplets was obtained after removing oil phase with hexane. SDS gel electrophoresis was performed to study the subunit of the protein layer using a vertical mini-gel system (Mini-PROTEIN Tetra Cell, BIO-RAD, Hercules, Calif., USA). Protein sample was mixed with the loading buffer (0.125 M Tris-HCl, pH 6.8, 4% (w/v) SDS, 20% (v/v) glycerol, 0.5% 2-mercaptoethanol and 1% bromophenol blue (w/v)) and then heated at 100° C. for 5 min. After cooling, 12 μL of sample (3 mg/ml) was loaded on a 5% stacking gel and 12% separating gel and subjected to electrophoresis at a constant voltage of 80 V. The gels were stained with 0.1% (w/v) Coomassie Brilliant Blue-R-250 in water-methanol-acetic acid (4:5:1, v:v:v) for 30 min and destained with water-methanol-acetic acid (4:5:1, v:v:v).

### Example 9

### Amino Acid Analysis

For amino acid analysis, the isolated protein coating was hydrolyzed under vacuum in 4 M methanesulfonic acid with 0.2% (w/v) tryptamine according to a modified method of Simpson et al. (1976). Glass sample tubes (6×50 mm) were used in the reaction vial assembly, which was then placed in the Work Station™ (Waters, Milford, Mass., USA) and treated according to the Work Station™ manual. The contents were hydrolyzed at 115° C. for 24 hr, followed by adjusting the pH to neutral with 3.5 M NaOH. Amino acid analysis was performed using the Waters ACCQ-Tag™ method. The high-performance liquid chromatography (HPLC) system (Agilent series 1100, Palo Alto, Calif., USA) consisted of an autosampler and a binary pump, a control system with a column heater maintained at 37° C., and a UV detector set at a wavelength of 254 nm. A reversed-phase ACCQ.Tag 150×3.9 mm C18 column with a solvent system consisting of a three-eluent gradient (ACCQ.Tag eluent, acetonitrile, and water) was used at a flow rate of 1.5 mL/miN. Data acquisition was controlled by a ChemStation™ software.

### Example 11

### Encapsulation Efficiency, Loading Efficiency and Moisture Content

Extraction of fish oil from the second example barley protein microcapsules was based on the method described by Beaulieu et al. (Beaulieu et al., 2002). Dry status microcapsules (250 mg) were weighed to the nearest 0.1 mg and added into 5 mL pure ethanol. The mixture was shaken on a vortex mixer for 1 min, the sample was allowed to rest for 5 min, and then 5 mL of hexane was added. The mixture was shaken vigorously with a vortex mixer for 30 s and allowed to stand for 2 min. These mixing and standing procedures were repeated twice. Five mL of water was added, and the tube was inverted several times, and then sealed and shaken using a Multi-purpose rotator (Barnstead 2314, IA, USA) for 1 h. After centrifugation (Beckman Coulter Avanti®J-E Centrifuge, Calif., USA) at 8,000×g for 15 min at 23° C., 4 mL of hexane was transferred to a tube and evaporated under nitrogen to remove the solvent. The remaining oil was weighed to the nearest 0.1 mg. The encapsulation efficiency (EE) and loading efficiency (LE) were calculated by the following equations: EE (%)=W_{encapsulated oil}/W_{total oil}×100; where W_{encapsulated oil} represents the weight of oil encapsulated in the microcapsule and W_{total oil} represents the oil added initially in the particle formation mixture. LE (%)=W_{encapsulated oil}/W_{microcapsules}×100; where W_{microcapsules} represents the weight of the microcapsule encapsulating the oil inside. The moisture content of the microcapsules was measured gravimetrically by drying ˜0.5 g of the dry status samples in an air oven at 105° C. for 12 h (Bae & Lee, 2008).

### Example 12

### Fish Oil Oxidative Stability in Accelerated Storage Test

The oxidative stability of the microencapsulated fish oil was tested at both dry status and in aqueous solutions (HCl-saline solution pH 2.0 and phosphate-buffered saline pH 7.0) at 40° C. for 8 weeks. For the stability test at dry status, approximately 5 g (dry weight) of each sample was placed in a pre-dried airtight glass container and stored in an incubator at 40° C. For the stability test at wet-status, approximately 5 g (dry weight) of freshly prepared microcapsules (without spray-drying) were suspended in pH 2.0 and 7.0 media, and incubated at 40° C. The oxidative stability was monitored by measuring the peroxide value (PV) of the extracted oils. Approximately 100 mg (dry weight) of each sample was withdrawn from the bottle at weekly intervals (Soottitantawat et al., 2005). The oil extraction process was the same as indicated above.

The colorimetric method described by Bae and Lee (2008) was used to measure the PV of oils with some modifications. The extracted oil (40 mg˜0.50 mg) was added to 9.8 mL of chloroform/methanol (7:3, v/v) mixture in a glass tube, followed by the addition of 50 μl each of ammonium thiocyanate and ferrous chloride solutions. The final mixture was then mixed and incubated for 5 min in a dimmed lit chamber at ambient temperature. After incubation, the absorbance was measured with a UV/vis spectrophotometer (model V-530, Jasco, Calif., USA) at 505 nm. Reagent and oil blank assays were also performed. PV was quantified in relation to a standard curve created from a series of hydrogen peroxide standard solutions and expressed as milliequivalents (meq) hydroperoxide per kg of oil.

### Example 13

### Fish Oil Stability in Selected Food Formulations (Milk and Yogurt)

The oxidative stability of the microencapsulated fish oil (wet status) was also tested in two food products. The microcapsule suspensions were mixed with milk or yogurt by stirring for 15 min to obtain homogeneous dispersions. These microcapsule-incorporated milk and yogurt were then pasteurized (80° C., 30 min) (Ng et al., 2011) and stored at 4° C. Sodium azide (0.025%, w/v) was added as a bacteriostatic agent. Samples were withdrawn weekly for fish oil stability analysis. The oil extraction process and the PV analysis were as described as above. The stability test was conducted for 4 and 5 weeks for milk and yogurt, respectively, according to their average shelf life. Original fat free milk and yogurt samples were used as zero controls.

### Example 10

### Statistical Analysis

All experiments were performed at least in triplicate. Each type of microcapsule was prepared in three independent batches. The microcapsule size, moisture content, EE and LE values were done in duplicate for each batch. Error bars on graphs represent standard deviations. Data is represented as the mean of three batches±SD. For each type of microcapsule, one batch of the sample was randomly selected for stability experiments. The PV data is the mean of three independent determinations±SD. Statistical significances of the differences were determined by Student's t-test. The level of significance used was p<0.05.

### REFERENCES

The following references are incorporated herein by reference (where permitted) as if reproduced in their entirety. All references are indicative of the level of skill of those skilled in the art to which this invention pertains.
Bae, E. K., & Lee, S. J. (2008). Microencapsulation of avocado oil by spray drying using whey protein and maltodextrin. Journal of Microencapsulation, 1-12.
Barrow, C. J., Nolan, C., & Holub, B. J. (2009). Bioequivalence of encapsulated and microencapsulated fish-oil supplementation. Journal of Functional Food, 38-43.
Beaulieu, L., Savoie, L., Paquin, P., & Subirade, M. (2002). Elaboration and Characterization of Whey Protein Beads by an Emulsification/Cold Gelation Process: Application for the Protection of Retinol. Biomacromol, 3, 239-248.
Beristain, C. I., Azuara, E., & Vernon-Carter, E. J. (2002). Effect of water activity on the stability to oxidation of spray-dried encapsulated orange peel oil using mesquite gum (Prosopis JuliXora) as wall material. Journal of Food Science, 67(1), 206-211.
Cauvain, S. P. (2003). Bread making: improving quality. CRC Press: Boca Raton.
Celus, I., Brijs, K. and Delcour, J. A. (2006) The effects of malting and mashing on barley protein extractability. Journal of Cereal Science 44:203-211.
Chen, L., Remondetto, G., & Subirade, M. (2006). Food protein-based materials as nutraceutical delivery systems. Trend Food Sci Tech, 17, 272-283.
Curtis, J. M., Berrigan, N., & Dauphinee, P. (2008). The Determination of n-3 Fatty Acid Levels in Food Products Containing Microencapsulated Fish Oil Using the One-Step Extraction Method. Part 1: Measurement in the Raw Ingredient and in Dry Powdered Foods. J Am Oil Chem Soc, 85, 297-305.
Davies, J. T., Shewry, P. R. and Harris, N. (1993) Spatial and temporal patterns of B hordein synthesis in developing barley (Hordeum vulgare L.) caryopses. Cell Biology International Reports 17:195-202.
Drusch, S., & Berg, S. (2008). Extractable oil in microcapsules prepared by spray-drying: Localisation, determination and impact on oxidative stability. Food Chemistry, 17-24.
Drusch, S., Serfert, Y., Heuvel, A. V. D., & Schwarz, K. (2006). Physicochemical characterization and oxidative stability of fish oil encapsulated in an amorphous matrix containing trehalose. Food Research International, 39, 807-815.
Drusch, S., Serfert, Y., Scampicchio, M., Hansberg, B. S., & Schwarz, K. (2007). Impact of Physicochemical Characteristics on the Oxidative Stability of Fish Oil Microencapsulated by Spray-Drying. J. Agric. Food Chem, 55, 11044-11051.
Drusch, S., & Schwarz, K. (2006). Microencapsulation properties of two different types of n octenylsuccinate-derivatised starch. European Food Research and Technology, 222, 155-164.
Eagles, H. A., Bedggood, A. G., Panozzo, J. F., & Martin, P. J. (1995). Cultivar and environmental effects on malting quality in barley. Australian Journal of Agricultural Research, 46, 831-844.
Finnie, C., & Svensson, B. (2009). Barley seed proteomics from spots to structures. Journal of Proteomics, 72, 315-325.
Firestone, D. (1993). Official Methods and Recommended Practices of the American Oil Chemist's Society. AOAC Press, Illinois.
Gharsallaoui, A., Roudaut, G., Chambin, O., Voilley, A., & Saurel, R. (2007). Application of spray-drying in microencapsulation of food ingredients: An overview. Food Research International, 40, 1107-1121.
Gibbs, B. F., Kermasha, S., Alli, I., & Mulligan, C. N. (1999). Encapsulation in food industry: A review. International Journal of Food Science and Food Nutrition, 50, 213-234.
Gouin, S. (2004). Micro-encapsulation: Industrial appraisal of existing technologies and trends. Trends in Food Science and Technology, 15, 330-347.
Hogan, S. A., Mcnamee, B. F., O'Riordan, E. D., & O'Sullivan, M. (2001). Microencapsulating Properties of Whey Protein Concentrate. J Food Sci, 66, 675-680.
Kagami, Y., Sugimura, S., Fujishima, N., Matsuda, K., Kometani, T., & Matsumura, Y. (2003). Oxidative stability, structure, and physical characteristics of microcapsules formed by spray drying of fish oil with protein and dextrin wall materials. Journal of Food Science, 68(7), 2248-2255.
Kanakdande, D., Bhosale, R., & Singhal, R. S. (2007). Stability of cumin oleoresin microencapsulated in different combination of gum arabic, maltodextrin and modified starch. Carbohydrate Polymers, 67(4), 536-541.
Kawase, S., Matsumura, Y., Murakami, H., & Mori, T. (1998). Comparison of antioxidative activity among three types of prolamin subunits. Journal of Cereal Science, 28, 33-41.
Keogh, M. K., Kennedy, B. T., Kelly, J., Auty, M. A., Kelly, P. M., Fureby, A., & Haahr, A. M. (2001). Stability to Oxidation of Spray-Dried Fish Oil Powder Microencapsulated Using Milk Ingredients. Journal of Food Science, 66, 217-224.
Kreis, M. and Shewry, P. R. (1989) Unusual features of cereal seed protein structure and evolution. Bioessays 10(6):201-7.
Krishnan, S., Bhosale, R., & Singhal, R. S. (2005). Microencapsulation of cardamom oleoresin: Evaluation of blends of gum arabic, maltodextrin and a modified starch as wall materials. Carbohydrate Polymers, 61(1), 95-102.
Le Meste, M., Champion, D., Roudaut, G., Blond, G., & Simatos, D. (2002). Glass transition and food technology: A critical appraisal. Journal of Food Science, 67, 2444-2458.
McMaster, T. J., Berry, M., Corfield, A. P. and Miles, M. J. (1999) Atomic force microscopy of the submolecular architecture of hydrated ocular mucins. Biophys J. 77(1):533-41.
Pan, X., Yao, P. and Jiang, M. (2007) Simultaneous nanoparticle formation and encapsulation driven by hydrophobic interaction of casein-graft-dextran and β-carotene. J. Colloid and Interface Science 315:456-463.
Naohiro, G., & Shun, W. (2006). The Importance of Peroxide Value in Assessing Food Quality and Food Safety. JAOCS, 83, 473-474.
Ng, E. W., Yeung, M., & Tong, P. S. (2011). Effects of yogurt starter cultures on the survival of Lactobacillus acidophilus. International Journal of Food Microbiology, 145, 169-175.
Partanen, R., Hakala, M., Sjovall, O., Kallio, H., & Forssell, P. (2005). Effect of relative humidity on the oxidative stability of microencapsulated sea buckthorn seed oil. Journal of Food Science, 70(1), 37-43.
Rich, G. T., Bailey, A. L., Faulks, R. M., Parker, M. L., Wickham, M. S. J. and Fillery-Travis, A. (2003a) Solubilization of carotenoids from carrot juice and spinach in lipid phases: I. Modeling the gastric lumen. Lipids 38(9):933-945.
Rich, G. T., Faulks, R. M., Wickham, M. S. J. and Fillery-Travis, A. (2003b) Solubilization of carotenoids from carrot juice and spinach in lipid phases: II. Modeling the duodenal environment. Lipids 38(9):947-956.
Rosenberg, M., & Sheu, T-Y. (1996). Microencapsulation of volatiles by spray-drying in whey protein-based wall systems. International Dairy Journal, 6(3), 273-284.
Simpson, D. L., Thorne, D. R. and Loch, H. H. (1976) Sulfated glycoproteins, glycolipids, and glycosaminoglycans from synaptic plasma and myelin membranes: isolation and characterization of sulfated glycopeptides. Biochemistry 15(25):5449-57.
Sheu, T. Y., & Rosenberg, M. (1998). Microstructure of microcapsules consisting of whey proteins and carbohydrates. Journal of Food Science, 63(3), 491-494.
Shewry, P. R., Franklin, J., Parmar, S., Smith, S. J., & Miflin, B. J. (1983). The effects of sulphur starvation on the amino acid and protein compositions of barley grain. Journal of Cereal Science, 1, 21-31.
Shewry, P. R., Kreis, M., Parmar, S., Lew, E. J. L., & Kasarda, D. D. (1985). Identification of g-type hordeins in barley. Federation of European Biochemical Societies Letters, 190, 61-64.
Shu, B., Yu, W., Zhao, Y., & Liu, X. (2006). Study on microencapsulation of lycopene by spray-drying. Journal of Food Engineering, 76, 664-669.
Soottitantawat, A., Bigeard, F., Yoshii, H., Furuta, T., Ohkawara, M., & Linko, P. (2005). Influence of emulsion and powder size on the stability of encapsulated D-limonene by spray drying. Innovative Food Science and Emerging Technologies, 6(1), 107-114.
Subirade, M., & Chen, L. (2008). Food-protein-derived materials and their use as carriers and delivery systems for active food components. In: Garti N, editor. Delivery and Controlled release of Bioactives in Foods and Nutraceuticals (pp. 251-278). Cambridge: Woodhead Publishing Ltd.
Wang, C., Tian, Z., Chen, L., Temelli, F., Liu, H. and Wang, Y. (2010) Functionality of barley proteins extracted and fractionated by alkaline and alcohol methods. Cereal Chem. 87(0):1-13.
Wang, R., Tian, Z., & Chen, L. (2011). Nano-encapsulations liberated from barley protein microparticles for oral delivery of bioactive compounds. International Journal of Pharmaceutics, 406, 153-162.
Wasaporn, C., Chockchai, T., & Shuryo, N. (2009). Antioxidative properties of partially purified barley hordein, rice bran protein fractions and their hydrolysates. Journal of Cereal Science, 49, 422-428.
Yal [symbol] in, E., [symbol] elik, S., & Ibano\check{g}lu, E. (2008). Foaming properties of barley protein isolates and hydrolysates. Eur Food Res Tech, 226, 967-974.

1. A microcapsule comprising a coating layer comprising barley protein, and a core comprising an oil.

2. The microcapsule of claim 1 having a size between about 3 μm to about 5 μm in diameter.

3. The microcapsule of claim 1, wherein the microcapsule has an encapsulation efficiency ranging between about 90% to about 100%.

4. The microcapsule of claim 1, wherein the microcapsule has a loading efficiency ranging between about 45% to about 50%.

5. The microcapsule of claim 1, wherein the coating consists essentially of hordein.

6. The microcapsule of claim 1 wherein the coating consists essentially of glutelin.

7. The microcapsule of claim 1 wherein the coating comprises hordein and glutelin in a pre-selected ratio.

8. The microcapsule of claim 1, wherein the microcapsule provides a zero-order or near zero-order release rate of the active ingredient in SIF plus pancreatin.

9. The microcapsule of claim 1, wherein the oil comprises a nut oil, or a vegetable oil, or a fish oil.

10. The microcapsule of claim 1 wherein the oil further comprises a biologically active ingredient.

11. The microcapsule of claim 10 wherein the active ingredient comprises an antibiotic, antiviral agent, non-steroidal anti-inflammatory drug, analgesic, hormone, growth factor, vitamin precursor, or vitamin.

12. The microcapsule of claim 11, wherein the active ingredient is beta-carotene.

13. The microcapsule of claim 1 which degrades to form nanoparticles comprising a protein and an oil droplet in a subject's stomach, and which further degrade to release the oil droplets in the subject's intestine.

14. A pharmaceutical or nutraceutical composition for treating, preventing or ameliorating a disease in a subject comprising a microcapsule as claimed in claim 1 in combination with one or more pharmaceutically acceptable carriers.

15. The composition of claim 14 which is a food or beverage.

16. The composition of claim 14 which is a dairy product.

17. A method of delivering a biologically active ingredient to a subject comprising orally administering to the subject in need thereof a microcapsule of claim 1 or a composition of claim 13, wherein said microcapsule is degraded to smaller but intact nanoparticles comprising a protein and an oil droplet in the stomach, which nanoparticles are then completely degraded in the intestine.

18. A method of treating, preventing or ameliorating a disease in a subject, or providing a physiological benefit or protection against a chronic disease, comprising orally administering to the subject in need thereof, a therapeutically effective amount of the microcapsule of claim 1 or the pharmaceutical composition of claim 12.

19. A method for preparing a barley protein encapsulated microcapsule, comprising the steps of:
a) blending an aqueous phase comprising barley protein and an oil to form a mixture;
b) emulsifying the mixture to form an emulsion; and
c) treating the emulsion to form microcapsules.

20. The method of claim 19 wherein the emulsion is treated in a microfluidizer or a homogenizer.
